(19) United States
(12) Patent Application Publication
(10) Pub. No.: US 2015/0338408 A1
(43) Pub. Date: Nov. 26, 2015

(54) SINGLE STEP CALIBRATION CURVE THROUGH SAMPLE CONVECTION

(71) Applicant: UNIVERSITY OF UTAH RESEARCH FOUNDATION, Salt Lake City, UT (US)

(72) Inventors: Marc D. Porter, Salt Lake City, UT (US); Michael C. Granger, Salt Lake City, UT (US); Nikola Pekas, Edmonton (CA)

(21) Appl. No.: 14/442,978

(22) PCT Filed: Nov. 14, 2013
(86) PCT No.: PCT/US13/70201

§ 371 Date: May 14, 2015

Related U.S. Application Data
(60) Provisional application No. 61/726,381, filed on Nov. 14, 2012.
(60) Provisional application No. 61/726,904, filed on Nov. 15, 2012.

(51) Int. Cl. G01N 33/569; G01F 1/00

(52) U.S. Cl. CPC G01N 33/56983; G01F 1/00; G01N 2333/015; G01N 2469/10

(57) ABSTRACT

This disclosure relates to methods and apparatuses for simultaneous measurement of multiple analytes in samples. This disclosure relates to methods and apparatuses for determining the concentration of one or more analytes in a fluid sample without use of a calibrant.

### CROSS-REFERENCE TO RELATED APPLICATIONS

This patent application claims priority to U.S. Provisional Patent Application No. 61/726,381 filed Nov. 14, 2012 and U.S. Provisional Patent Application No. 61/726,904 filed Nov. 15, 2012, the contents of which are incorporated herein by reference in their entirety.

### STATEMENT REGARDING FEDERALLY SPONSORED RESEARCH

This invention was made with government support under grants 1 U01 CA151650-01 and 1 R33 CA155586-01A1 awarded by the National Institutes of Health. The government has certain rights in the invention.

### FIELD

This disclosure relates to methods and assemblies for simultaneous quantitative measurement of multiple analytes in samples. This disclosure relates to methods and apparatuses for determining the concentration of one or more analytes in a fluid sample without use of a calibrant.

### INTRODUCTION

Efforts in personalized medicine involve simultaneous measurement of multiple analytes in a sample from a patient. One approach to measure these multiple analytes is with arrays or chips with addresses, with one address for each capture reagent to bind an analyte. Accurate quantification of the individual analytes in unknown samples requires measurement of known analyte concentrations to generate calibration curves. As the number of tested analytes increases, it becomes increasingly difficult and cumbersome to establish a calibration curve or standard for each analyte.

### SUMMARY

In an aspect, the disclosure relates to an apparatus for measuring the amount of one or more analytes in a sample. The apparatus may include a surface including an area for receiving the sample, the area designed to accept fluid convection of a fluid sample, and having a first set of locations for measuring the amount of a first analyte in the sample, wherein at least two of the locations of the first set of locations experiences a different flux of first analytes by virtue of its placement on said receiving area. In some embodiments, the first set of locations comprises a plurality of discrete locations positioned in the area and arranged in a first line extending away from the center point, each location in the first set comprising a first capture reagent bound to the surface and capable of specifically binding to the first analyte. The apparatuses may include a means of providing fluid convection of the sample to the receiving area and plurality of discrete locations. In some embodiments, the apparatus further comprises a second set of locations for measuring the amount of a second analyte in the sample, the second set comprising a plurality of discrete locations positioned in the area and arranged in such a way to receive a different flux of the second analyte each location in the second set comprising a second capture reagent bound to the surface and capable of specifically binding to the second analyte. In some embodiments, the second line is at a non-zero angle relative to the first line.

In a further aspect, the disclosure relates to methods for determining the concentration of one or more analytes in a sample. The methods may include applying the sample to the area of the apparatus described herein for a selected period of time in a manner that induces fluid convection, detecting at each location a signal from a detection label associated with each analyte, wherein the signal at each location corresponds to the amount of the analyte at each location, and determining the concentration of each analyte in the sample as a function of the signals detected at each location, the rate of fluid convection, the position of the location, and the selected period of time. The notion of rotation described herein is an example of fluid convection of sample to a surface designated to receive the sample, however, other mechanisms of fluid convection, such as liquid jet impingement and transport through induced vortices, are implied through phenomenological similarity

In yet another aspect, the disclosure relates to methods of determining the concentration of one or more analytes in a fluid sample without use of a calibrant. The methods may include inducing fluid convection of the sample in a system adapted to capture the analyte at a plurality of distinct locations thereby inducing a flux of the analyte, wherein the amount of analyte captured at each distinct location is proportional to the flux of the analyte at that location, wherein the flux at a first location is different than the flux at a second location; predicting the flux of the analyte at each of the first and second locations; determining the concentration of analyte in the sample by correlating the detected signals to the predicted fluxes using the concentration of the analyte as the sole correlating parameter. The fluid convection may comprise forced convection, natural convection, buoyant convection, granular convection, thermomagnetic convection, capillary action, the Managoni and Weissenberg effects, combustion, or a combination thereof. The flux may be predicted using one or more of equations governing fluid convection, equations governing analyte mass transport, and ligand-receptor dynamics. The equations that govern fluid convection comprise the convection-diffusion equation, the Navier-Stokes equations, and the Euler equations, wherein the equations that govern mass transport comprise the Nernst-Planck equation, the Buckely-Leverett equation, Darcy's Law, Fick's laws of diffusion, and the Maxwell-Stefan equation, and wherein the ligand-receptor dynamics can be described by kinetic and thermodynamic treatments of chemical equilibria. The convection consists of advection or diffusion. The system may comprise a surface including an area for receiving the sample, the area designed to accept fluid convection of the sample, and a plurality of capture reagents located at each of the distinct locations. Each of the distinct locations may comprise a defined address. The methods may further comprise determining the concentration of a second analyte in the fluid sample without use of a calibrant.

The disclosure provides for other aspects and embodiments that will be apparent in light of the following detailed description and accompanying Figures.

### BRIEF DESCRIPTION OF THE DRAWINGS

FIG. 1 is an idealization of the capture and labeling of antigen for immunometric assay: (A) immobilization of capture monoclonal antibody (mAb); (B) preparation of label antibody and reporter (Extrinsic Raman Label or ERL); and (C) antigen capture and labeling steps; (D) Schematic of biochip; (E) Surface Enhanced Raman Spectroscopy (SERS) response of a blank and immobilized feline calcivirus (FCV); (F) the corresponding 5×5 μm Atomic Force Microscopy (AFM) image of ERLs bound to immobilized FCV.

FIG. 2(A) is a graph of the number of porcine parvovirus (PPV) bound to the capture substrates at varying rotation rates. The solid lines are weighted fits of the experimental data to Equation 5. FIG. 2(B) is a single-address biochip schematic depicting the three fluid velocities realized when a surface is rotated in a solution. FIG. 2(C) is a plot of the theoretical dependence of equilibrium surface concentration of the antigen-antibody complex (Γ_{AgAb}) on address location relative to the body center. FIG. 2(D) is a schematic of capture address with diameter of 2β located at a remote location (r₀) from the center of a rotating substrate.

FIG. 3(a) is an image of a surface (or coupon) according to the invention with alternating Ni and Au addresses. FIG. 3(b) is an image of the back of the surface shown in FIG. 3(a), showing an adhesive magnetic sponge for securing the surface during rotation. FIG. 3(c) is an image of a polyether ether ketone (PEEK) holder for the surface of FIGS. 3(a) and 3(b), the holder having a press fit magnetic disk. FIG. 3(d) is an image of the surface of FIGS. 3(a) and 3(b) secured in the holder of FIG. 3(c).

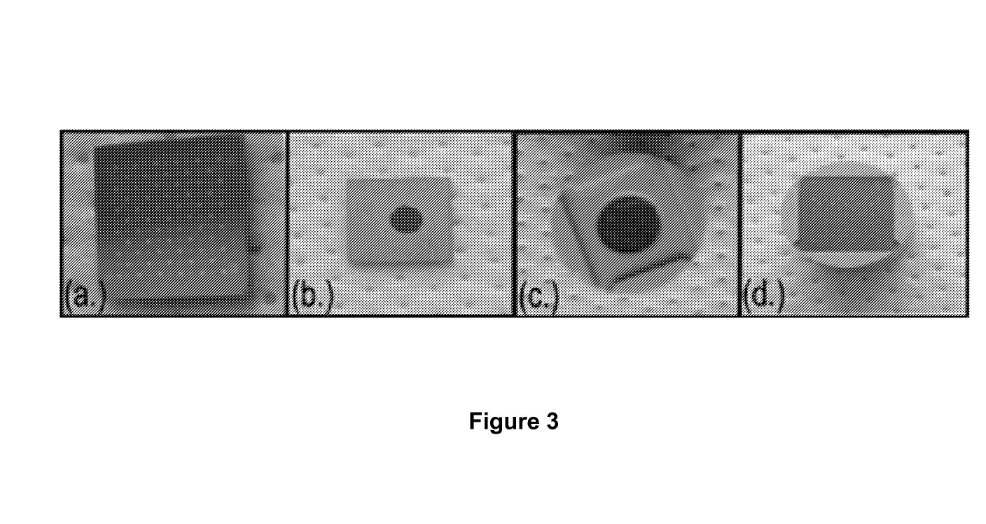

FIG. 4 shows the realistic 3D model of a polyether ether ketone (PEEK) rotator with a surface (or coupon) according to the invention affixed to its surface.

FIG. 5 shows the radial surface concentration of a dilute species after exposing the model of FIG. 4 to the dilute species and rotating the model for 10 minutes at 250 rpm. The smoothed line is a fit of the computed data.

FIG. 6 shows a radial dependence of addresses that selectively bind Human Ostopontin (OPN) after exposing a rotating address to OPN at varying radii.

FIG. 7(a) shows a schematic of one means of inducing fluid convection according to the present invention that utilizes geometrical or hydrodynamic “necking” to alter and confine the flow of analyte. The addresses experience a differential flux as the sample flows in the direction shown. FIG. 7(b) is a graph showing the relative flux as a function of address number corresponding to the schematic of FIG. 7(a). FIG. 7(c) is an image of induced fluid convection of fluorescein in a system according to the schematic of FIG. 7(a). FIG. 7(d) is a simplified vector calculus representation of the convection-diffusion equation.

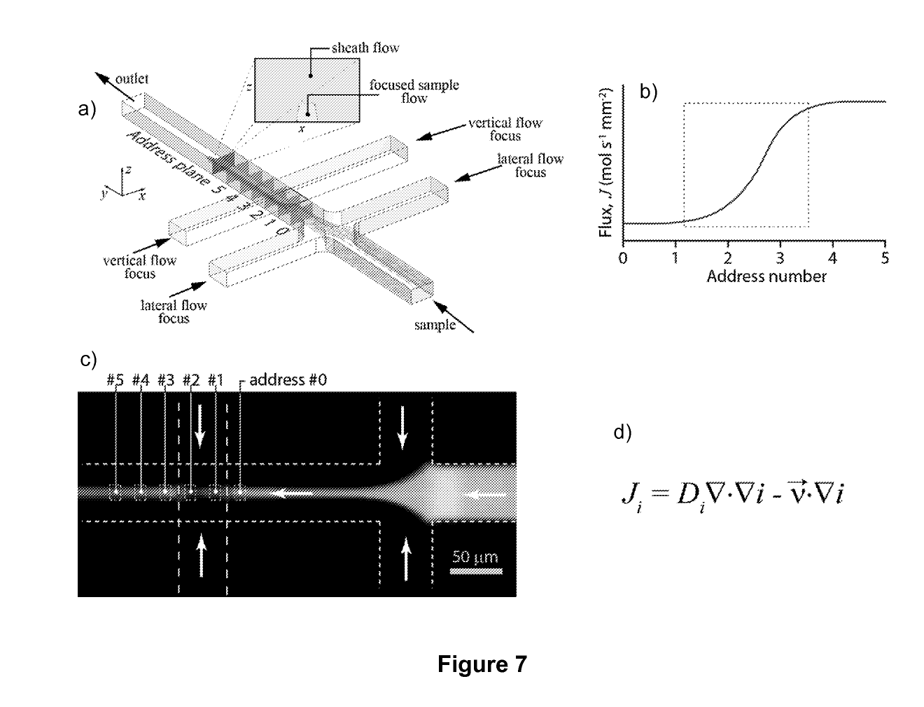

FIG. 8 shows a schematic of one means of inducing fluid convection according to the present invention that utilizes capillary fluid flow. The addresses experience a differential flux as fluid flows in the direction shown.

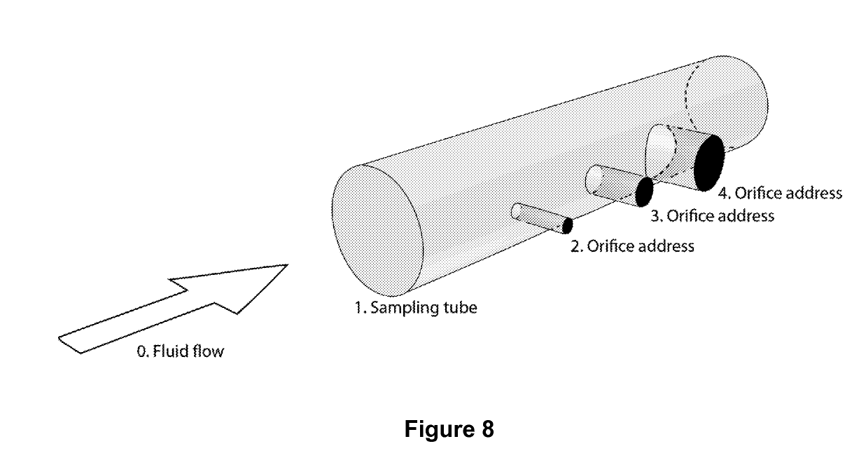

### DETAILED DESCRIPTION

The methods and systems disclosed herein are not limited in their applications to the details of construction and the arrangement of components described herein. The methods and apparatuses are capable of other embodiments and of being practiced or of being carried out in various ways. Also it is to be understood that the phraseology and terminology used herein is for the purpose of description only, and should not be regarded as limiting. Ordinal indicators, such as first, second, and third, as used in the description and the claims to refer to various structures, are not meant to be construed to indicate any specific structures, or any particular order or configuration to such structures. The use of the terms “a” and “an” and “the” and similar referents in the context of describing the invention are to be construed to cover both the singular and the plural, unless otherwise indicated herein or clearly contradicted by context. All methods described herein can be performed in any suitable order unless otherwise indicated herein or otherwise clearly contradicted by context. The terms “comprising,” “having,” “including,” and “containing” are to be construed as open-ended terms (i.e., meaning “including but not limited to”) unless otherwise noted. Embodiments described as “comprising” certain features are also contemplated as “consisting essentially of” and “consisting of” said features unless otherwise noted. The use of any and all examples, or exemplary language (e.g., “such as”) provided herein, is intended merely to better illuminate the methods and apparatuses disclosed herein and does not pose a limitation on the scope of the methods and apparatuses unless otherwise claimed. No language in the specification, and no structures shown in the drawings, should be construed as indicating that any non-claimed element is essential to the practice of the methods and apparatuses disclosed herein.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration, volume or the like range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this application.

Further, no admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein.

This disclosure provides apparatuses and methods for measuring the amount or determining the concentration of one or more different types of analytes in a fluid sample.

This disclosure provides methods for determining the concentration of one or more analytes in a fluid sample without use of a calibrant. The methods may comprise one or more of the following step: inducing fluid convection of the sample in a system adapted to capture the analyte at a plurality of distinct locations thereby inducing a flux of the analyte, wherein the amount of analyte captured at each distinct location is proportional to the flux of the analyte at that location, wherein the flux at a first location is different than the flux at a second location; predicting the flux of the analyte at each of the first and second locations; detecting a signal from each of the first and second locations; and determining the concentration of analyte in the sample by correlating the detected signals to the predicted fluxes using the concentration of the analyte as the sole correlating parameter. In certain embodiments, the fluid convection may be selected from the group consisting of forced convection, natural convection, buoyant convection, granular convection, thermomagnetic convection, capillary action, the Managoni and Weissenberg effects, combustion, and combinations thereof.

Suitable surfaces for use with the apparatuses and methods disclosed herein may include any surface capable of containing addresses as described herein and whose properties are sufficiently understood to enable a person having ordinary skill in the relevant art to predict the fluid dynamics of a fluid contacting the surface.

Suitable surfaces may include, for example, conductive, semiconductive and nonconductive surfaces, surface-modified and non-surface-modified surfaces, and the like. Examples of surface materials include, but are not limited to, semiconductors (e.g., Si, GaAs, GaN, Ge, and CdSe, among others), polymers (e.g., acrylic, polystyrene, rubber, nylon, silicones, polyurethanes, siloxanes, and epoxies, among others), photosensitive polymers—positive and negative resists (e.g., phenol-formaldehyde resins [i.e., Novolac photoresists], methacrylates, and benzocyclobutenes, among others), glass (e.g., Pyrex® and borosilicate, among others), oxides/ceramics/insulators (e.g., SiO₂, In₂O₃, and SnO, among others), nitrides (e.g., Si₃N₄, among others), carbon (e.g., highly oriented pyrolytic graphite [HOPG], glassy carbon, and graphene, among others), quartz, metal (e.g. gold, magnetic metals, such as nickel, iron and cobalt, and metals having surface tension-induced pumping action, such as mercury, among others), living tissue, and plated cell cultures, among others.

The surface may include an area for receiving a fluid sample. The area may be designed to accept fluid convection of a fluid sample.

A location or address may be a discrete sub-area on the surface or within the area of the surface. A location may contain one or more addresses. An address may comprise at least one capture reagent. A location or address may be defined by the presence of more or less capture reagents in the sub-area as compared with the region immediately surrounding the location or address (e.g., the location or address contains capture reagents while the immediately surrounding region does not) or may be defined by the presence of a means of probing the presence of an analyte in the sub-area as compared with the region immediately surrounding the location or address (e.g., the location or address is defined by a laser spot where the laser serves a means of probing the presence of the analyte). In certain embodiments, the locations or addresses are defined by the presence of capture reagent as compared with a surrounding surface that contains essentially no capture reagent. In certain embodiments, the surface is uniformly covered by capture reagent and the locations or addresses are defined by interrogating a specific area of the surface. The effect is that the presence of an analyte is probed only within a known area or volume for comparison with the predicted flux of analyte for the known area or volume.

An address may comprise the same material as the surface or may comprise a different material than the surface. Suitable address materials include any material capable of being derivatized to contain capture reagents as described herein. Suitable addresses may include, for example, conductive, semiconductive and nonconductive surfaces, surface-modified and non-surface-modified surfaces, and the like. Examples of address materials include, but are not limited to, semiconductors (e.g., Si, GaAs, GaN, Ge, and CdSe, among others), polymers (e.g., acrylic, polystyrene, rubber, nylon, silicones, polyurethanes, siloxanes, and epoxies, among others), photosensitive polymers—positive and negative resists (e.g., phenol-formaldehyde resins [i.e., Novolac photoresists], methacrylates, and benzocyclobutenes, among others), glass (e.g., Pyrex® and borosilicate, among others), oxides/ceramics/insulators (e.g., SiO₂, In₂O₃, SnO, and ZnO, among others), nitrides (e.g., Si₃N₄, among others), carbon (e.g., highly oriented pyrolytic graphic [HOPG], glassy carbon, and graphene, among others), quartz, metal (e.g. gold, magnetic metals, such as nickel, iron and cobalt, metals having surface tension-induced pumping action, such as mercury, among others), living tissue, and plated cell cultures, among others.

Locations or addresses may be of any size that affords suitable distinction of flux when compared with at least one other location or address. Suitable locations or addresses may have an area that can be reproducibly interrogated. Suitable locations or addresses may have sufficient area to contain enough capture reagents that exposure to analytes in the concentrations described herein for the lengths of time described herein does not saturate the capture reagents. In some embodiments, the location or address may have an area of at least about 100 nm², at least about 500 nm², at least about 1 μm², at least about 5 μm², at least about 10 μm², at least about 50 μm², at least about 100 μm², at least about 150 μm², at least about 200 μm², at least about 250 μm², at least about 300 μm², at least about 350 μm², at least about 400 μm², at least about 450 μm², at least about 500 μm², at least about 550 μm², at least about 600 μm², at least about 650 μm², at least about 700 μm², at least about 750 μm², at least about 800 μm², at least about 850 μm², at least about 900 μm², at least about 950 μm², at least about 1 mm², at least about 2 mm², at least about 3 mm², at least about 4 mm², at least about 5 mm², at least about 6 mm², at least about 7 mm², at least about 8 mm², at least about 9 mm², at least about 10 mm², at least about 15 mm², at least about 20 mm², at least about 25 mm², at least about 50 mm², at least about 75 mm², at least about 100 mm², at least about 500 mm², or at least about 1 cm². In some embodiments, the address may have an area of at most about 1 m², at most about 100 cm², at most about 50 cm², at most about 10 cm², at most about 5 cm², at most about 1 cm², at most about 500 mm², at most about 100 mm², at most about 90 mm², at most about 80 mm², at most about 70 mm², at most about 60 mm², at most about 50 mm², at most about 40 mm², at most about 30 mm², at most about 20 mm², at most about 10 mm², at most about 9 mm², at most about 8 mm², at most about 7 mm², at most about 6 mm², at most about 5 mm², at most about 4 mm², at most about 3 mm², at most about 2 mm², or at most about 1 mm², at most about 900 μm², at most about 800 μm², at most about 700 μm², at most about 600 μm², at most about 500 μm², at most about 400 μm², at most about 300 μm², at most about 200 μm², at most about 100 μm², at most about 90 μm², at most about 80 μm², at most about 70 μm², at most about 60 μm², at most about 50 μm², at most about 40 μm², at most about 30 μm², at most about 20 μm², or at most about 10 μm².

In certain embodiments, the location or address may be two-dimensional (i.e. binding to a surface). In certain embodiments, the location or address may be three-dimensional (i.e. binding within a volume). In embodiments where the location or address is three-dimensional, the location or address may suitably have a volume that encompasses the values disclosed herein for suitable areas raised to a power of 3/2.

The locations or addresses may have any geometric shape. In certain embodiments, a location or address may have the shape of a circle, a triangle, or a quadrilateral, among others. In certain embodiments, a location or address may have the shape of a sphere, a tetrahedron, or a cube, among others.

In certain embodiments, a set of locations or addresses may comprise at least two locations or addresses arranged to experience a different flux of analytes by virtue of their placement on the surface or in an area on the surface. In certain embodiments, the set of locations or addresses may comprise at least three, at least four, at least five, at least six, at least seven, at least eight, at least nine, at least ten, or any integer number of locations or addresses arranged to experience a different flux of analytes by virtue of their placement on the surface or in an area on the surface. In principle, a set of locations or addresses may comprise as many addresses as can be physically located on the surface or area on the surface.

In certain embodiments, the surface or area may include one, two, three, four, five, six, seven, eight, nine, ten or any integer number of sets of locations or addresses. In certain embodiments, each set of locations or addresses corresponds to a unique analyte whose concentration may be determined by the apparatuses and methods disclosed herein. In principle, the surface or area may include as many sets of locations or addresses as can be physically fit on the surface or area.

Suitable capture reagents for use with the apparatuses and methods disclosed herein may include any capture reagent capable of being located within an address (e.g., capable of being affixed to an address surface or within an address volume) and capable of selectively binding an analyte. Examples of capture reagents include, but are not limited to, antibodies, polynucleotides, polypeptides, aptamers, imprinted polymers, proteins, species capable of binding an analyte by virtue of hybridization or base pairing (i.e., single stranded DNA/RNA, micro RNA (miRNA), and the like), chelating agents (e.g., EDTA), binding hosts (e.g., crown ethers, porphyrins, pthalocyanines, and cyclodextrins, among others). In certain embodiments, where there is no need to identify the analyte, the capture reagent may comprise a size exclusion filter element.

Suitable analytes for use with the apparatuses and methods disclosed herein may include any analyte of interest capable of undergoing fluid convection within a fluid and capable of being selectively bound by a capture reagent as disclosed herein. Suitable analytes include those whose properties are sufficiently understood to enable a person having ordinary skill in the relevant art to predict the fluid dynamics of the analyte in the apparatuses and methods.

Examples of analytes include, but are not limited to, organic molecules or particulates, inorganic molecules or particulates, biologic entities (alive, viable, non-viable, dead, etc.), biomolecules (e.g., polypeptides, carbohydrates, glycoproteins, cytokines, hormones, proteins, cells, viruses, spores, small chemical compounds, and large chemical compounds, among others), single cell organisms, genetic material, volatile organic materials, and colloidal or micellar entities, among others. In certain embodiments, the analyte may comprise a material germane to biomarkers (i.e., materials that provide an ability to detect/prognose/stratify a health state).

In certain embodiments, the analyte may require a detection label in order to be identified, quantified or a combination thereof. In embodiments requiring a detection label, the detection label may be introduced to the surface in the same fashion that the analyte was introduced to the surface or by any other method known to a person having ordinary skill in the relevant art.

In certain embodiments, the analyte may comprise an intrinsic label.

In certain embodiments, the analyte may change a property of the capture reagent, thus enabling detection via measurement of the change. In certain embodiments, the capture reagent may contain an intrinsic label that is released upon selective binding of the analyte, wherein the presence of analyte is indicated by a reduction in signal from the intrinsic label.

Suitable detection labels for use with the apparatuses and methods disclosed herein my include any detection label capable of selectively binding an analyte as disclosed herein and being interrogated to identify, quantify, or identify and quantify the analyte to which the detection label is bound.

Suitable detection labels may include, for example, extrinsic Raman labels, fluorophores, chromophores, chemiluminescent labels, molecular beacons, fluorescence resonance energy transfer probes, molecular zippers, and colorimetric labels by virtue of enzymatic substrate turnover (e.g., ELISA labeling technique), among others.

Suitable fluids for use with the apparatuses and methods disclosed herein may include any fluid capable of containing an analyte of interest and whose properties are sufficiently understood to enable a person having ordinary skill in the relevant art to predict the fluid dynamics of the fluid in the apparatuses and methods.

Suitable fluids may include, for example, liquids, gases, plasmas, and the like. Examples of fluids include, but are not limited to, aqueous fluids, organic solvents, inorganic solvents, patient tissue lysates, bodily fluids, phosphate buffered saline, buccal material, viral transport media, biologically relevant buffer systems (e.g., HEPES, MOPS, MES, and DEPC, among others), chromatographic mobile phases, marine environment samples (e.g., seawater, pond water, river water, and lake water, among others), blood, ocular fluid, cerebral spinal fluid, diluents, liquid metals, inert gases, atmospheric gases, aqueous vapors, organic vapors, inorganic vapors, and gaseous metals, among others.

Fluid convection may be induced by any suitable method known to a person having ordinary skill in the relevant art. In certain embodiments, fluid convection may be induced, for example, by any individual item or combination thereof from the following non-limiting list: i) a surface having an area as described herein may be rotated and introduced to the fluid sample; ii) free jet impingement of the sample; iii) an increased solution linear velocity may be realized in a microfluidic channel as a function of narrowing cross section—as flux to the surface is proportional to linear velocity, the increased velocity as a function of narrowing cross section may induce a differential flux; iv) an off-axis secondary flow (e.g., a sheath-type flow) for bolus specific change in velocity may be repeated for many boluses with each bolus having a distinct velocity—and hence, a distinct flux (this option could alternatively be used to localize different amounts of analyte near a no-slip boundary for instances where laminar behavior is observed); v) elevating addresses throughout velocity lamina in non-turbulent systems (e.g., through the parabolic flow profile in Poiseuille flow); vi) “dragging” the substrate through solution at varying velocity (e.g., by using optical tweezers); vii) inducing capillary flow in a series of capillaries each having different surface chemistries, diameters, or a combination thereof, to realize disparate velocities and corresponding differential flux (suitable for use in lateral flow assay format); viii) a thermal gradient for predictable changes in diffusivity and kinematic viscosity or diescrete (zoned) changes for address specific convective behavior changes; ix) applying electric field(s) to impose migration gradients; and x) applying magnetic field combined with electric field to give rise to electromagnetic, or Lorentzian, flow for address-specific changes in flux (e.g., applying periodic orthogonal AC-current in a constant magnetic field, which is parallel to the direction of fluid flow, produces a periodic Lorentzian force capable of moving precise volumes of solution over capture addresses).

In certain embodiments, inducing fluid convection in a system may induce a subsequent predicted differential flux of analytes between a plurality of distinct locations within the system. In other words, inducing fluid convection may induce a differential flux that is capable of being predicted by methods known to a person having ordinary skill in the relevant art.

In certain embodiments, the predicted differential flux is predicted by one or more of equations governing fluid convection, equations governing analyte mass transport, and ligand-receptor dynamics. Examples of equations governing fluid convection include, but are not limited to, the convection-diffusion equation, the Navier-Stokes equations, and the Euler equations, among others. Examples of equations governing analyte mass transport include, but are not limited to, the Nernst-Planck equation, the Buckely-Leverett equation, Darcy's Law, Fick's laws of diffusion, and the Maxwell-Stefan equation, among others. Examples of ligand-receptor dynamics include, but are not limited to, kinetic and thermodynamic treatments of chemical equilibria, among others.

In certain embodiments, the methods disclosed herein may comprise determining conditions for inducing fluid convection. Suitably, determining conditions for inducing fluid convection may comprise use of the same equations as set forth for predicting the predicted flux.

Fluid convection can be induced in a variety of forms. In certain embodiments, fluid convection may comprise forced convection, natural convection, buoyant convection, granular convection, thermomagnetic convection, capillary action, the Managoni and Weissenberg effects, combustion, or a combination thereof. In certain embodiments, convection may comprise advection or diffusion.

In certain embodiments, the methods disclosed herein may comprise detecting a signal from at least two distinct locations having differential flux of analytes.

In certain embodiments, the methods disclosed herein may comprise determining the concentration of each analyte in the sample as a function of the signals detected as each address. In certain embodiments, to determine the relationship between the concentration and signals, application of Navier-Stokes equations to geometries or embodiments described herein may result in analytical expressions or numerical solutions describing hydrodynamic behavior; from this approach—which may be performed using finite elemental analysis modeling approaches—expressions containing concentration can be developed in which the only fitting parameter that is allowed to persist is concentration.

In certain embodiments, the methods disclosed herein may comprise determining the concentration of analyte in the sample by correlating signal to predicted flux using analyte concentration as the sole correlating parameter. Using this approach, the predicted flux can be fit to the signals solely by varying the analyte concentration. When the best fit has been achieved, the concentration that corresponds to that fit is the determined concentration. In general, correlating may be performed by any method known to a person having ordinary skill in the relevant art. In some embodiments, the determining step is subsequent to detecting a signal.

As the benefits of a many-marker panel-approach in in vitro diagnostics become clearer and the need to accurately quantify multiplexed markers in screening studies increases, the ability to accurately and simultaneously determine multiple analyte concentrations becomes even more urgent. In today's diagnostic microarray platforms, absolute analyte level determination is accomplished by simultaneously running a calibration series for each analyte, which is an untenable approach when considering simultaneous analysis of hundreds of analytes from single samples (analyte multiplexing).

The forthcoming revolution in medicine involves simultaneous measurement of multiple analytes (disease markers) in patient tissue lysates and bodily fluids (blood, serum, plasma, sputum, urine, etc.). Evidence pointing to the power of a multi-analyte (panel) approach includes a 90-95% effective early pregnancy test for Trisomy-21. From a detection rate below 40%, the combination of a six-biochemical marker panel and two physical markers with an empirical algorithm gave the highest sensitivity for prenatal detection of chromosomal defects, while maximizing specificity, according to this test. The use of differential protein profiling has also lent credence to multi-marker approaches to diagnose infectious diseases, neurodegenerative diseases, and cancers. The concept of regular and simultaneous monitoring of multiple markers is not only gaining acceptance in the diagnostics arena but is also an important core component of personalized medicine, which is a medical model in which systematic use of information pertaining to the gene, protein, and metabolite profiles of an individual can be used to specifically tailor medical care. However, the ability to identify health risk, disease susceptibility, and response to therapy remains largely unreachable due in part to our technical inability to easily screen a single sample for the large number of biomarkers that potentially make up each wellness map.

For the nascent panel-approach paradigm to take hold and flourish, there are several key factors to consider: (1) identification of accurate disease and wellness markers and understanding the implications of up- and down-regulation of these markers; (2) the ability to accurately quantitate the markers at all disease stages; (3) implementation of facile patient sampling procedures; and (4) placing easy-to-use, low-cost, and extensible assay technology in the hands of healthcare providers. The scope of this disclosure focuses on factor (2): the development of a calibration method that addresses the need to accurately quantify potentially hundreds of markers simultaneously.

A convenient, cost-effective assay platform might consist of sample chips with multiple spotted addresses, each for specific analyte capture. However, accurate quantification of each analyte in an unknown sample may require measurement of known analyte concentrations to calibrate the detection platform. The highly parallel nature of such multiplexed analyses presents a problem in designing devices that use today's technology to create multiple calibration curves (e.g., pipetting a dilution series of calibrants and controls into specific wells of a microtiter plate for each analyte), the cornerstone in quantitative marker analysis. An alternative but similarly inadequate calibration approach relying on methods embedded in today's microarray paradigm may require sequential marker analyses, which may reduce the benefit of a multiplexed analyte platform.

The compositions and methods described herein fulfill the need for a highly efficient calibration method for multiplexed analyte analysis. In one embodiment, controlled sample convection may be used to simultaneously determine absolute solution concentration for each marker studied.

To realize this calibration scheme, a large number of capture addresses (analogous to wells in a microtiter plate format) may be used. Small antigen-capture addresses may be used to facilitate the large number of capture addresses. This decrease in address size also contributes to the overall capture efficiency and limit of detection (LOD) of the assay by concentrating captured markers. The development of this concept is summarized through Equations 1-3 for a 1:1 binding reaction between a solution phase antigen, Agₛₒᵢₙ, and immobilized antibody, Ab_{surf}, to form a surface bound antigen-antibody complex, AgAb_{surf} (Equation 1).

$$\begin{matrix}{[ Ag ]}_{sol} + {[ Ab ]}_{surf} ⇔ {[ AgAb ]}_{surf} & ( 1 ) \\ K = \frac{{[ AgAb ]}_{surf}}{{{[ Ag ]}_{sol} [ Ab ]}_{surf}} & ( 2 ) \\ {Γ}_{AgAb} = ( {C}_{i} - {[ Ag ]}_{sol} ) \frac{V}{A} & ( 3 )\end{matrix}$$

The equilibrium constant, K (L/mol), for the reaction is defined by Equation 2, wherein [AgAb]_{surf} (mol/cm²) and [Ab]_{surf} (mol/cm²) are the respective equilibrium surface concentrations of the antigen-antibody complex and uncomplexed capture antibody, and [Ag]ₛₒₗ (mol/L) is the equilibrium concentration of antigen. Since the equilibrium involves a liquid and solid phase, a full accounting of the system includes the area of the capture substrate, A, and sample volume, V. The equilibrium surface concentration of the antigen-antibody complex, Γ_{AgAb} (mol/cm²), at equilibrium can then be written per Equation 3, where Cᵢ is the initial concentration of antigen. Equation 3 indicates that if V and Cᵢ are held constant, Γ_{AgAb} is inversely proportional to A. Thus, a decrease in the size of the capture address by a factor of 100 translates to a 100× increase in Γ_{AgAb}. This also indicates that the LOD will be lowered by 100× if, as confirmed below, the concentrating nature of the decrease in address size does not increase nonspecific adsorption (NSA). Lastly, through mass balance considerations, defining Γ_{Ab,i} as the initial surface concentration of capture antibody, and substituting Equation 3 into Equation 2, the following expression is obtained (Equation 4), which can be used to probe the impact of experimental variables like K, V, A, Cᵢ, and Γ_{Ab,i} on Γ_{AgAb}:

KAΓ_{AgAb}²+(−V−KCᵢV−KAΓ_{Ab,i})Γ_{AgAb}+KCᵢVΓ_{Ab,i}=0 (4)

The other aspect of the universal calibration method for multiplexed analyte analysis is hydrodynamic enhanced delivery (forced convection) or increased flux of Ag (antigen or analyte) and label. This can be accomplished through rotating the capture address in the sample and labeling solution. Though the methods and compositions described herein may be used for single analyte analysis, multi-analyte calibration is also possible. Alternate forms of forced sample convection could be substituted for rotation with similar outcomes.

Both theoretically and experimentally it is apparent that Γ_{AgAb}, as a function of time and rotation rate, is given by Equation 5:

$$\begin{matrix}{Γ}_{AbAg} = \frac{2}{{π}^{1 / 2}} {D}^{1 / 2} {Ct}^{1 / 2} + \frac{{D}^{2 / 3} C}{1.6 {lv}^{1 / 6}} t {ω}^{1 / 2} & ( 5 ) \\ {Γ}_{AgAb} = a + b {ω}^{1 / 2} & ( 6 )\end{matrix}$$

where D is the antigen diffusion coefficient, C is the solution concentration, t is time, ν is solution kinematic viscosity, and ω is rotation rate. The first term in Equation 5 represents the diffusional contribution to mass transfer; the second term defines the hydrodynamically enhanced mass transfer via substrate rotation. There are three assumptions, all experimentally validated, central to the derivation of this equation: (1) the reactant solution concentration is independent of binding; (2) the binding sites at the surface do not saturate; and (3) the recognition reaction is fast compared to the delivery of reactant. Equation 5 describes how binding can be manipulated by varying t and, more importantly, ω. Using this system, the diffusion coefficient can be determined if Γ_{AgAb} and C are known; conversely, if Γ_{AgAb} and D are known, C can be determined by fitting the curve to Equation 6 (a simplified version of Equation 5). That is, simply fitting a plot of Γ_{AgAb} vs. ω, allows one to determine the value of C. For use with real-world readout platforms, once an initial correlation of Γ_{AgAb} and the transduced signal (e.g., fluorescence) is performed, a plot of signal vs. ω will yield C.

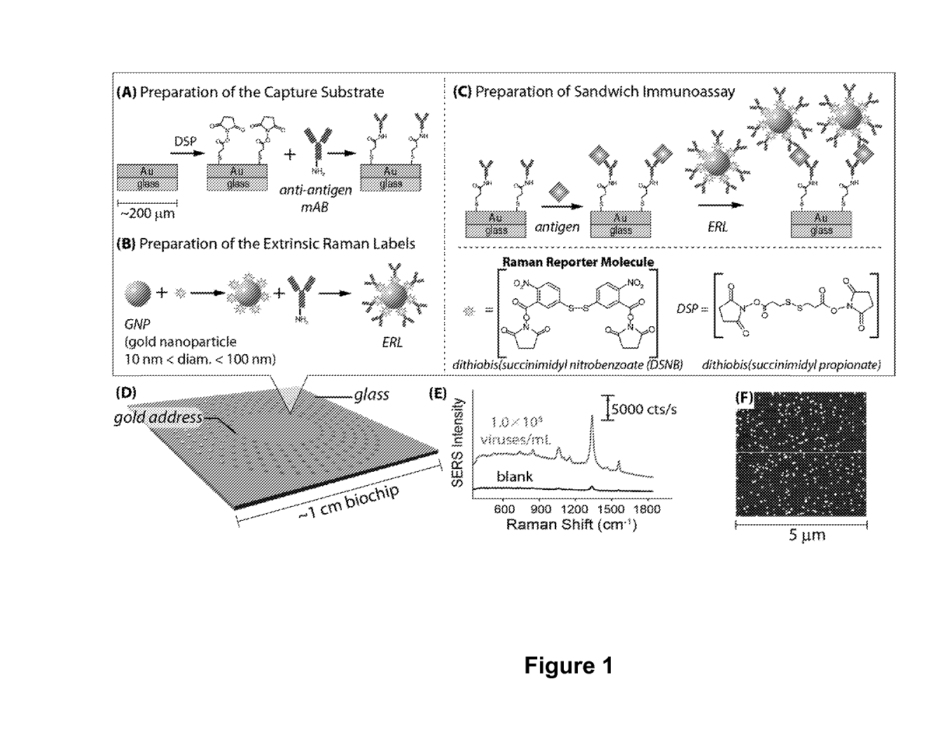

To accurately ascribe a concentration to a specific marker (also termed analyte and antigen herein), a method to calibrate the measuring platform for that marker may be used. In conventional diagnostic microarray platforms, this is accomplished by screening a number of samples for the same marker in the same microtiter plate while simultaneously running a calibration series for that marker (high sample throughput). This method allows for multiplexed sample analysis; however, it is impractical for multiplexed marker analysis. For example, when running a panel of 20 markers for a single sample, 100 wells would be used to create 20 five-point calibration curves. Even using cutting-edge automated fluid handling platforms, this calibration method quickly becomes untenable as the number of markers increases above several tens of markers. Described herein is a series of experiments that provide methods of multiplexed marker calibration: the use of sample convection to ultimately yield an absolute solution concentration for each marker studied. Our test-bed for these studies is the chip-scale format microarray immunoassay. Depicted in FIG. 1(A-C) is an idealized cartoon detailing the stepwise derivatization and proposed use of the microarray biochip for the studies proposed herein. Though antibodies are depicted as the capture reagent, capture reagents may include, but are not limited to, antibodies, polynucleotides, and polypeptides, aptamers, imprinted polymers, or proteins. Likewise, analytes or antigens include a wide variety of biomolecules such as, for example, polypeptides, carbohydrates, glycoproteins, cytokines, hormones, proteins, cells, viruses, spores, and other small and large chemical compounds.

The solid phase biochip, a so-called “capture substrate” version, is shown in FIG. 1(D). Immunorecognition is imparted to the biocbip by covalently linking specific capture monoclonal antibodies (mAbs) to lithographically-derived gold addresses. The assay is completed in four steps: sample application to the biochip; analyte capture by the chip; labeling the captured analyte; and readout. The biochip is prepared with a large array of analyte capture sites—gold addresses—each one able to capture a different analyte. Capture occurs through immunorecognition, i.e., an address-confined capture monoclonal antibody (mAb) with high affinity for a specific analyte is able to capture its complimentary analyte, or antigen (Ag). This occurs when sample is applied to the biochip, and if present, analyte is captured at its complimentary address. The captured analyte is then labeled with secondary, or label mAbs, which are prepared by attaching them to a reporter. For this work the reporter takes the form of an Extrinsic Raman Label (ERL; FIG. 1(B)), which is used to generate a surface-enhanced Raman scattering (SERS) spectrophotometric signal (representative spectra of our previous work focused on ultrasensitive detection of feline calicivirus (FCV) are shown in FIG. 1(E)) and can easily be counted by atomic force microscopy (AFM; FIG. 1(F)) or scanning electron microscopy (SEM) when enumeration is required. The results of the calibration studies proposed herein can be applied to any readout method; they are not SERS or AFM specific. For example, a biochip scheme may be used to analyze one analyte per biochip and a calibration curve for that analyte may be constructed from several biochips, each exposed to a different analyte concentration. When combined with a small address footprint and forced fluid convection, ultrasensitive assays for a variety of analytes may be realized.

### EXAMPLES

### Example 1

### Preliminary Data

To test the impact of a decrease in A (area of the capture address), we prepared 0.040 mm² (200×200 μm) capture addresses and compared their function to capture addresses 3-mm in diameter (7.1 mm²). Both address sizes were then modified with a mAb for porcine parvovirus (PPV) and incubated for 24 h in 1.0 mL of stagnant PPV diluted in phosphate buffered saline (PBS). Captured PPV was then labeled with ERLs also coated with the same mAb. Dose-response curves were prepared which, after accounting for NSA in the blanks, showed an improvement in LOD of ˜100× for the smaller addresses (LOD: 4×10⁵ PPV/mL, ˜750 aM) versus the 3-mm addresses (LOD: 4×10⁷ PPV/mL, −70 fM). Based on this and other similar results in our previous work, we believe that a decrease in address size will provide a biochip capable not only of accommodating the sheer number of addresses needed for multi analyte calibration but also of decreasing LOD for each analyte.

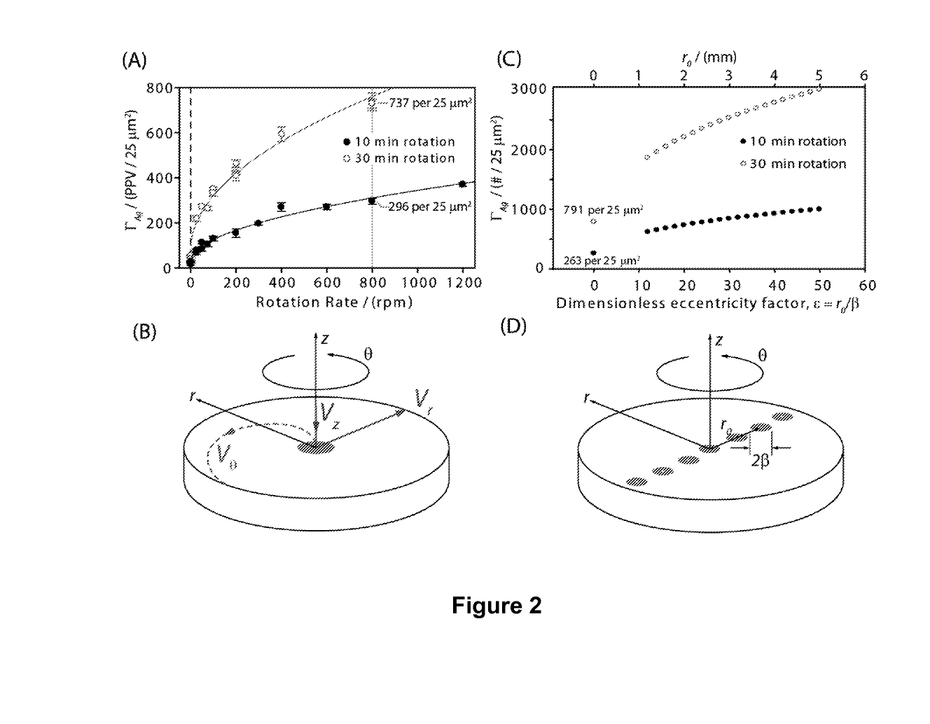

The theoretical implications of forced rotationally-induced convection through a number of empirical studies were confirmed. The results of a representative study that employed PPV as a model system are shown in FIG. 2(A). These data were acquired by rotating a capture substrate, depicted schematically in FIG. 2(B), at a singular w and measuring the number of bound PPV, determined by AFM enumeration of 5×5 μm areas. A new capture substrate was used for each point plotted in FIG. 1(A). The solid line is the fit to experimental data using Equation 6 (D=1.75×10⁷ cm²/s, v=1.004×10⁻² cm²/s at 25° C., and t=30 or 10 min) which yielded a bulk concentration, C, of PPV=4.6×10⁹ PPV/mL. It is important to note that these data were obtained by immersing multiple single-address capture substrates in a solution for rotation. However, a simpler experimental method can be employed to determine C if the substrate has multiple addresses, each located at a distinct distance from the center of the rotating substrate, designated r₀ in FIG. 2(D). Though the radial (Vᵣ) and perpendicular flow (V_{z}) is constant across the surface, every address will experience a different tangential flow (V_{θ} in FIG. 2(B)), or flux of analyte, and hence a different amount of marker accumulation. This dependence of accumulation on r₀ (or more precisely ε, the so-called eccentricity factor (ε=r₀/β) which is valid when r₀>>β) is given by Equation 7:

Γ_{AgAb}=0.64D^{2/3}ω^{1/2}tC*ν^{-1/6}ε^{1/3} (7)

This dependence can then be used to construct a plot of r_{AgAb} vs. r₀ to determine C for each analyte from a single biochip (FIG. 2(C)), which is analogous to the method of Γ_{AgAb} vs. w without having to use a multitude of capture substrates. If sufficient addresses are present and series of those addresses are dedicated to specific analytes, one could determine the concentration of multiple analytes simultaneously by rotating a single biochip in one sample. A single biochip could be used to determine multiple biomarker concentrations from a single sample aliquot.

### Example 2

### Sample Convection for Calibration

Rotational investigations were performed in one well of a 48-well micro titer plate using a rotator with controllable and highly accurate rotation rates. The holder containing a biochip was lowered into the well and rotated for a specified time at a specific rotation rate. The biochip consists of an optically flat 100-mm Pyrex wafer (0.5 mm thick) lithographically patterned with Au addresses using conventional lithographic techniques and diced to provide 10-mm square biochips containing 200×200 μm addresses arrayed in either a circular or orthogonal pattern to provide replicate addresses at distinct r₀ for each analyte (FIG. 1(D). By using arrayed addresses of this size the strengths of rotation and small address area are combined as detailed above. The biochip addresses were derivatized with capture mAb.

Once the fundamental issues of cross reactivity and NSA have been ameliorated, calibration studies will commence. A single biochip will be rotated for a specified duration and rate in both sample and labeling solution; individual wells will be used for each solution. Between the two rotations, the biochip will be rotated in a rinse buffer. The first sets of samples will be prepared by quantitative addition of single and mixtures of analytes in PBS. This series of studies is designed to optimize the assay for rotation rate and duration in each of the three wells and will be compared to theoretical predictions. The completed biochip will be gently blown dry with high purity nitrogen and analyzed by SERS, SEM, and AFM as a function of r₀. Plots analogous to the theoretical plot of FIG. 2(C) will be prepared to ascertain the concentration of each analyte and the effectiveness of this approach to provide a calibration method for multiplexed analyte analysis. The optimized set of procedures will then be employed for an analogous study in which the sample matrix is replaced with commercially available pooled rabbit serum.

Flux gradients, not following predicted behavior, may occur; conditions will be monitored for this phenomenon and if it is encountered, efforts may be made to create a more uniform rotating fluid volume. In the same vein, convective shear forces may successfully compete for antigen and label or deteriorate the immobilized capture-Ab surface. This possibility will be mitigated through careful monitoring of signal evolution and control of angular velocity. The outcome of these studies is also intimately tied to the affinity constant of each mAb-Ag interaction; if K values from analyte to analyte are too disparate, a complete dose response profile may not be realized for each analyte during the course of multiplexed analysis. Conditions will be monitored for this eventuality and Γ_{Ab,i} will be altered to account for discrepancies in K. Any changes in sample viscosity will be accounted for by use of a single exogenous internal standard introduced to the sample prior to inducement of convection.

The multi-analyte calibration methods proposed herein may fill the need of a highly efficient calibration method for multiplexed analyte analysis that will ultimately bring a panel-approach paradigm for disease diagnosis closer to reality.

### Example 3

### 3D Realistic Model

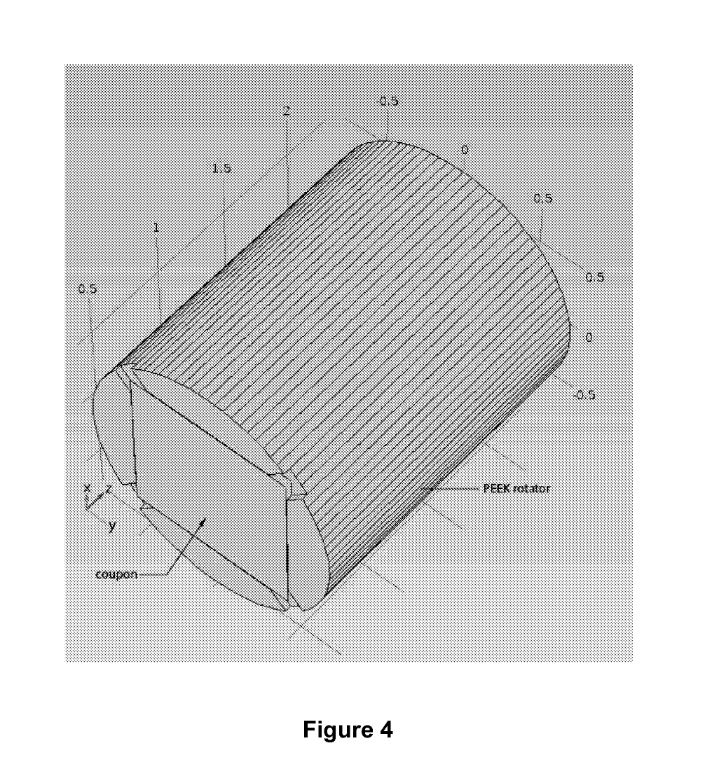

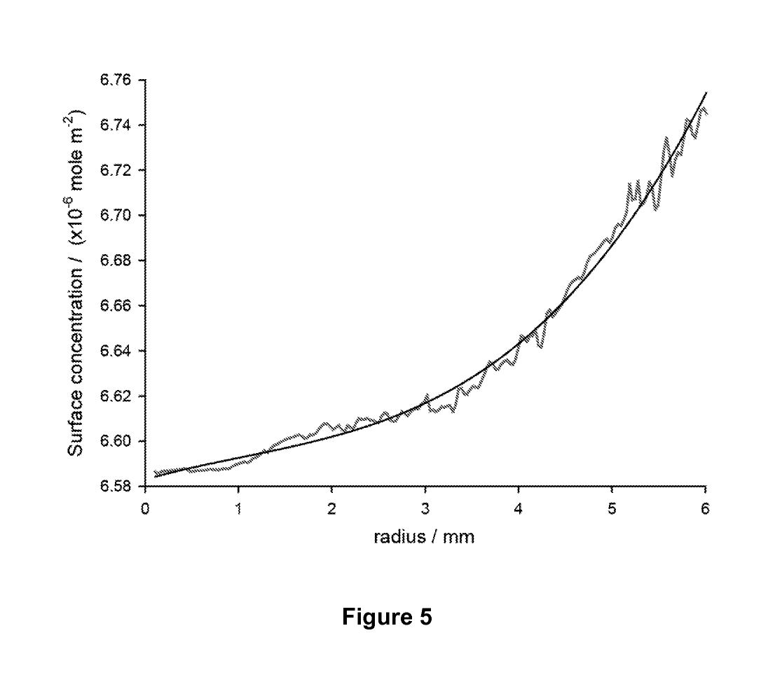

As shown in FIG. 4, a 3D realistic model of a polyether ether ketone (PEEK) rotator with a surface according to the present invention affixed thereto was prepared for use in fluid dynamic and analyte flux studies. Dimensions in FIG. 4 are in centimeters. The behavior of contacting the surface of FIG. 4 with a dilute species (1×10⁻³ mol/m³) while rotating the surface at 250 rpm for 10 minutes is shown in FIG. 5. The computed data are shown with a third order polynomial fit (smoothed line). As can be seen, an increase in surface concentration is observed as a function of radius, which constitutes the realization of predicted flux.

### Example 4

### Determining OPN Concentration

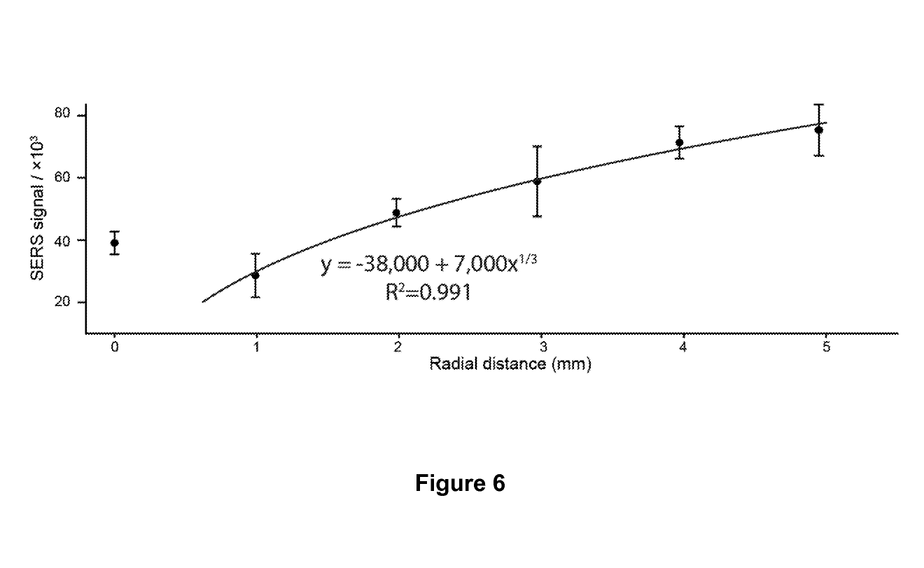

A single address having capture reagents that selectively bind Human Osteopontin (OPN) was exposed to a 1 ng/mL OPN solution and rotated at varying radial distances at 300 rpm for 10 minutes. In between exposures, the address was rinsed with a 10 mM NaCl borate or phosphate buffer. The data are reported in FIG. 6. With the exception of the center point, these data demonstrate a cube root dependence on radius. The equation for the fit is shown as an inset of FIG. 6. The lack of fitting compliance observed for the center address (radius of 0 mm) was expected as the fluidic profile at the center of a rotating disk does not follow the same radial, tangential, and normal fluidic components of the majority of the disk. The observed radial dependence of signal is a seminal result that confirms our ability to deduce sample marker concentrations without the use of calibrants, greatly simplifying quantitative multiplexed marker analysis.

1. An apparatus for measuring the amount of one or more analytes in a fluid sample, the apparatus comprising:
a surface including an area for receiving the sample, the area designed to accept fluid convection of the sample; and
a first set of locations for measuring the amount of a first analyte in the sample, the first set comprising a plurality of discrete locations positioned in the area and at least two of the locations of the first set arranged to experience a different flux of first analytes by virtue of their placement in the area, each discrete location in the first set comprising a first capture reagent bound to the surface and capable of specifically binding to the first analyte.

2. The apparatus of claim 1, further comprising a means of providing fluid convection of the sample to the receiving area and plurality of discrete locations.

3. The apparatus of claim 1, wherein the plurality of discrete locations of the first set extends radially in a first line from a center point of the area.

4. The apparatus of claim 1, further comprising a second set of locations for measuring the amount of a second analyte in the sample, the second set comprising a plurality of discrete locations positioned in the area and at least two of the locations of the second set arranged to experience a different flux of second analytes by virtue of their placement in the area, each discrete location in the second set comprising a second capture reagent bound to the surface and capable of specifically binding to the second analyte.

5. The apparatus of claim 4, wherein the plurality of discrete locations of the first set extends radially in a first line from a center point of the area, and wherein the plurality of discrete locations of the second set extends radially in a second line from a center point of the area.

6. The apparatus of claim 5, wherein the second line is at a non-zero angle relative to the first line.

7. A method for determining the concentration of one or more analytes in a sample, the method comprising:
applying the sample to the area of the apparatus of claim 1 for a selected period of time in a manner that induces fluid convection;
detecting at each location a signal from a detection label associated with each analyte, wherein the signal at each location corresponds to the amount of the analyte at each location; and
determining the concentration of each analyte in the sample as a function of the signals detected at each location, the rate of fluid convection, the position of the location, and the selected period of time.

8. The method of claim 7, wherein fluid convection is induced by rotating the area about an axis of rotation at a selected rotation rate for a selected period of time.

9. The method of claim 7, wherein fluid convection is induced using free liquid jet impingement of the sample.

10. The method of claim 7, wherein fluid convection is induced by transporting the sample through induced vortices.

11. A method of determining the concentration of an analyte in a fluid sample without use of a calibrant, the method comprising:
inducing fluid convection of the sample in a system adapted to capture the analyte at a plurality of distinct locations thereby inducing a flux of the analyte, wherein the amount of analyte captured at each distinct location is proportional to the flux of the analyte at that location, wherein the flux at a first location is different than the flux at a second location;
predicting the flux of the analyte at each of the first and second locations;
detecting a signal from each of the first and second locations; and
determining the concentration of analyte in the sample by correlating the detected signals to the predicted fluxes using the concentration of the analyte as the sole correlating parameter.

12. The method of claim 11, wherein fluid convection comprises forced convection, natural convection, buoyant convection, granular convection, thermomagnetic convection, capillary action, the Managoni and Weissenberg effects, combustion, or a combination thereof.

13. The method of claim 11, wherein the flux is predicted using one or more of equations governing fluid convection, equations governing analyte mass transport, and ligand-receptor dynamics.

14. The method of claim 13, wherein the equations that govern fluid convection comprise the convection-diffusion equation, the Navier-Stokes equations, and the Euler equations, wherein the equations that govern mass transport comprise the Nernst-Planck equation, the Buckely-Leverett equation, Darcy's Law, Fick's laws of diffusion, and the Maxwell-Stefan equation, and wherein the ligand-receptor dynamics can be described by kinetic and thermodynamic treatments of chemical equilibria.

15. The method of claim 11, wherein the convection consists of advection or diffusion.

16. The method of claim 11, wherein the system comprises:
a surface including an area for receiving the sample, the area designed to accept fluid convection of the sample; and
a plurality of capture reagents located at each of the distinct locations.

17. The method of claim 16, wherein each of the distinct locations comprises a defined address.

18. The method of claim 11, further comprising determining the concentration of a second analyte in the fluid sample without use of a calibrant.
